(19) United States
(12) Patent Application Publication
(10) Pub. No.: US 2024/0341614 A1
(43) Pub. Date: Oct. 17, 2024

(54) Imaging Systems and Imaging Methods

(71) Applicant: Battelle Memorial Institute, Richland, WA (US)

(72) Inventors: Richard Trevor Clark, West Richland, WA (US); David M. Sheen, Richland, WA (US)

(73) Assignee: Battelle Memorial Institute, Richland, WA (US)

(21) Appl. No.: 18/133,749

(22) Filed: Apr. 12, 2023

(51) Int. Cl. A61B 5/0507; A61B 8/08

(52) U.S. Cl. CPC A61B 5/0507; A61B 8/52

(57) ABSTRACT

Imaging systems and imaging methods are described. According to one aspect, an imaging system includes processing circuitry configured to access radar data resulting from reflection of electromagnetic energy from a target imaging volume, first focus the radar data to provide first focused data for at least substantially an entirety of the target imaging volume, use the first focused data to identify a sub-volume of the target imaging volume, and second focus the radar data to provide second focused data for the sub-volume of the target imaging volume, and wherein the second focused data has increased resolution compared with the first focused data and the second focused data comprises an image of the target imaging volume.

### STATEMENT AS TO RIGHTS TO INVENTIONS MADE UNDER FEDERALLY-SPONSORED RESEARCH AND DEVELOPMENT

This invention was made with Government support under Contract DE-AC05-76RL01830 awarded by the U.S. Department of Energy. The Government has certain rights in the invention.

### TECHNICAL FIELD

This disclosure relates to imaging systems and imaging methods.

### BACKGROUND OF THE DISCLOSURE

Screening of personnel for concealed weapons has become increasingly important as threats to aviation and other public venues, such as public arenas and courthouses, have evolved. The security posture at airports has been driven by high-profile events. Screening systems have been primarily directed towards detecting objects, such as weapons, explosives, etc. which are concealed upon and under clothing of individuals. Initial security focus was directed to detecting concealed handguns and knives and metal detectors for passenger screening and x-ray systems for hand-carried baggage and items were largely enough to mitigate threats.

Radar imaging technology using microwave and millimeter-wave (MMW) electromagnetic energy has been shown to detect concealed weapons of individuals because these signals are able to penetrate common clothing materials and are amenable to precise mathematical focusing techniques. However, microwave and MMW image reconstruction including mathematically focusing data is very computation intensive. Methods using faster reconstruction speeds enable lower latency and/or higher resolution but also may result in non-ideal artifacts in the results.

At least some of the aspects of the disclosure are directed to imaging systems and methods that have increased computational efficiency compared with some conventional systems and methods. According to some aspects described herein, radar data is focused at different resolutions to increase the speed of the focusing while maintaining optimal fidelity of generated images. Additional aspects of the disclosure are discussed in the example embodiments below.

### BRIEF DESCRIPTION OF THE DRAWINGS

Example embodiments of the disclosure are described below with reference to the following accompanying drawings.

FIG. 1 is an illustrative representation of an imaging system according to one embodiment.

FIG. 2 is a block diagram of components of an imaging system according to one embodiment.

FIG. 3 is an illustrative representation of a unit cell according to one embodiment.

FIG. 4 is an illustrative representation of a multistatic scanned aperture imaging system according to one embodiment.

FIG. 5A is a three-dimensional fine voxel grid of a target imaging volume according to one embodiment.

FIG. 5B is a three-dimensional coarse voxel grid of a target imaging volume according to one embodiment.

FIG. 6A is an image resulting from coarse scanning of a target imaging volume according to one embodiment.

FIG. 6B is a dilated version of the image of FIG. 6A according to one embodiment.

### DETAILED DESCRIPTION OF THE DISCLOSURE

This disclosure is submitted in furtherance of the constitutional purposes of the U.S. Patent Laws “to promote the progress of science and useful arts” (Article 1, Section 8).

Some aspects of the disclosure are directed towards apparatus, systems and methods for detecting weapons or other objects which may be concealed, for example under clothing of a person or within other objects. At least some of the described imaging systems and methods may be utilized in varied applications to scan a three-dimensional (3D) target imaging volume including a person and generate radar images of scatterers within the target imaging volume as a result of the scanning. The generated images may be used to detect concealed weapons or objects.

In cases of scanning a person or other object, the resultant radar images are sparse, or in other words, the 3D volume only has meaningful data in a small percentage of the voxels. According to some of the embodiments described herein, the speed of processing of radar data resulting from a scan of the target imaging volume may be increased by an order of magnitude or more by processing the radar data at different resolutions.

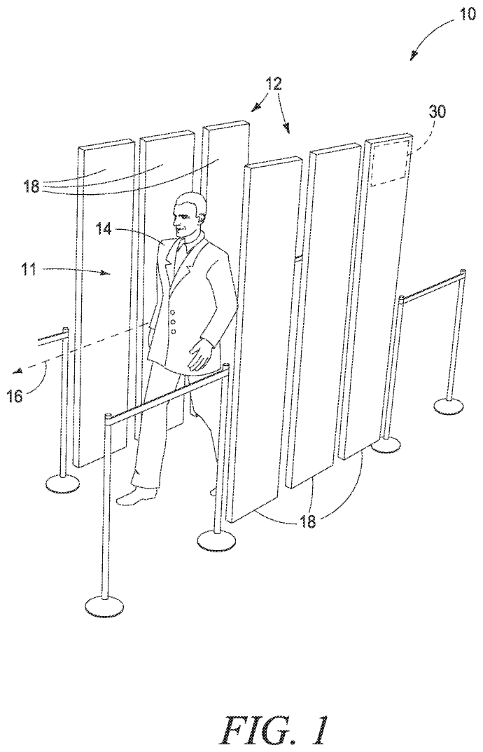

Referring to FIG. 1, an example imaging system 10 is shown according to one embodiment. The system 10 may be installed in one example application to provide threat detection at points of ingress/egress of a public facility. The depicted example imaging system 10 includes plural antenna systems 12 that are positioned opposite one another about a target imaging volume 11. Each of the antenna systems 12 includes a plurality of antenna arrays 18. The antenna arrays 18 are configured as columns in the depicted antenna system 12 having a multi-column architecture. The antenna arrays 18 each include a plurality of transmit antennas that are configured to emit electromagnetic energy towards target imaging volume 11 and a plurality of receive antennas that are configured to receive electromagnetic energy reflected from scatterers within the target imaging volume 11 during scanning and imaging operations (the transmit and receive antennas are not shown in FIG. 1).

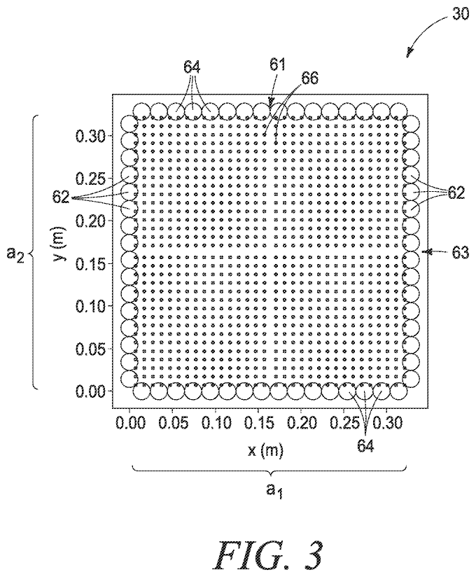

In one embodiment, each of the arrays 18 incudes a plurality of unit cells 30. In one more specific embodiment, each antenna array 18 includes six unit cells 30 that are arranged vertically where the unit cells 30 are positioned elevationally above one another along the vertical extent of the respective antenna array 18. Additional details of an example unit cell 30 are shown in FIG. 3 and discussed below. Other arrangements for implementing transmit and receive antennas of the antenna system 12 are possible, for example including only one array system 12 or different numbers of arrays 18 or unit cells 30 of transmit and receive antennas that may be arranged in other configurations such as horizontally. Additional details regarding operations of the antenna system 12 during scanning are discussed below.

A person 14 to be screened enters the imaging system 10 which attempts to detect the presence of concealed objects, such as weapons or explosives, upon the person 14 as they walk in a direction 16 through the target imaging volume 11 or stand within volume 11. The antenna systems 12 each emit electromagnetic energy towards the person 14 in the target imaging volume 11 and receive electromagnetic energy reflected from the person 14.

As discussed below, the received electromagnetic energy may be focused to provide information regarding one or more items that may be concealed upon the person 14. The results of the electromagnetic scanning and focusing may be used in one or more ways, such as the generation of images of the target imaging volume including the person 14. The images may be displayed via a computer monitor, processed using automated threat detection or artificial intelligence, and/or stored for subsequent processing and use in example implementations. The use of two antenna systems 12 permits scanning of two sides of the person 14 as the person 14 moves through, or stands within, the imaging system 10 and the target imaging volume 11.

In some embodiments, the two systems 12 transmit at slightly different times or frequencies to reduce interference therebetween. The transmit/receive channels on a single system 12 may be sequenced to only have one transmitter active at a time with one or more simultaneous receivers. In one other embodiments, the transmitters may be sequenced to minimize or eliminate interference through spatial and/or time multiplexing.

In the described example embodiment, the antenna systems 12 are stationary and do not move during scanning operations. Each of the antenna systems 12 are configured to scan a two-dimensional (2D) aperture to implement multistatic scanning of the target imaging volume 11 where different combinations of transmit and receive antennas are selected to emit and receive electromagnetic energy at different moments in time. The transmit and receive antennas are selected at different moments in time to provide raster scanning of a plurality of different sampling points across substantially the entire 2D aperture during the scanning as discussed further below.

In other embodiments, an antenna array may be physically moved to scan a 2D aperture during scanning operations, for example as discussed in U.S. patent application Ser. No. 17/959,890, filed Oct. 4, 2022, titled “Imaging Systems and Associated Methods”, having inventors David M. Sheen and Richard Trevor Clark, the teachings of which are incorporated herein by reference.

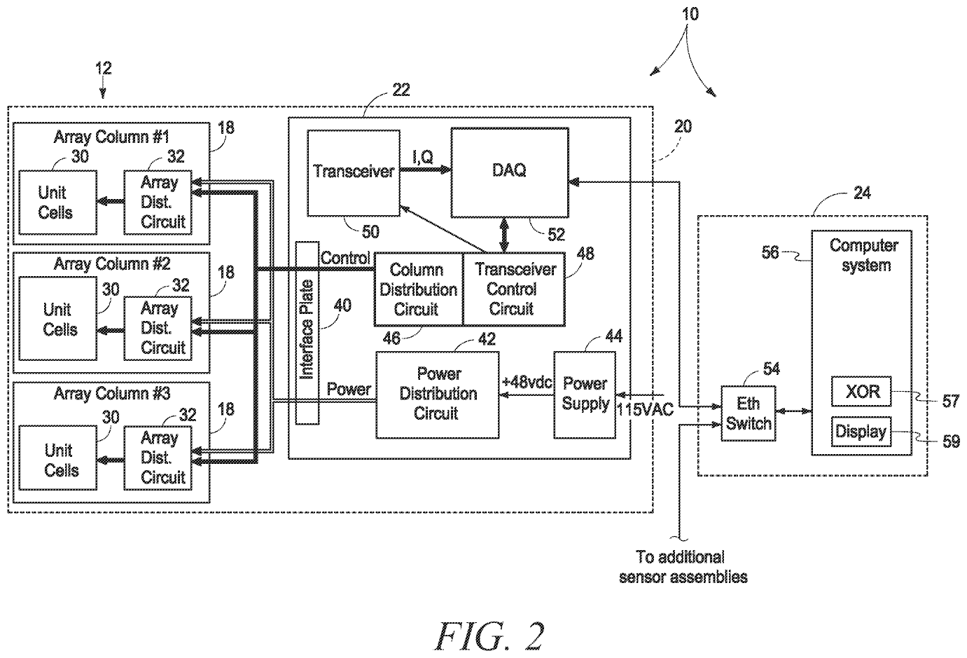

Referring to FIG. 2, a functional block diagram of components of an example embodiment of imaging system 10 are shown. The illustrated imaging system 10 includes one or more sensor assemblies 20 (each including one of the antenna systems 12) and an operator work station 24 in the illustrated embodiment. The depicted sensor assembly 20 includes plural array arrays 18 of the antenna system 12 and an associated electronics system 22 as shown.

The individual arrays 18 include a plurality of unit cells 30 and an array distribution circuit 32. The array distribution circuit 32 routes RF transmit and receive channels to desired transmit and receive antenna combinations of the respective unit cell 30.

Electronics system 22 includes an interface plate 40, a power distribution circuit 42, a power supply 44, a column distribution circuit 46, a transceiver control circuit 48, a transceiver 50, and a data acquisition system (DAQ) 52.

Interface plate 40 provides a bulkhead for electrical connections between the electronics system 22 and the antenna arrays 18 and provides operational power from power distribution circuit 42 and power supply 44 to antenna arrays 18. In addition, interface plate 40 communicates control signals from column distribution circuit 46 to antenna arrays 18 to control the transmission of electromagnetic energy from the antenna arrays 18 and receives signals from the antenna arrays 18 corresponding to reflections of electromagnetic energy from the target imaging volume.

Power distribution circuit 42 receives operational electrical energy from power supply 44 and distributes the electrical energy at appropriate voltages to the antenna arrays 18 via interface plate 40. Power supply 44 receives AC electricity (e.g., 115 VAC) and outputs DC electricity (e.g., +48 VDC) to power distribution circuit 42.

Column distribution circuit 46 includes logic to select different pairs of antennas of the antenna arrays 18 during scanning operations and may be referred to as a controller. Transceiver control circuit 48 is configured to control operations of transceiver 50 including the generation and application of signals therefrom to the transmit antennas to emit electromagnetic energy from the antenna arrays 18.

Some embodiments of the imaging system are based upon a frequency modulated continuous wave (FMCW) voltage-controlled oscillator (VCO) driven transceiver architecture. For example, transceiver control circuit 48 controls the transceiver 50 to control the transmission or emission of electromagnetic energy in a sweep of a desired frequency range capable of penetrating clothing to identify concealed items in one embodiment. In one specific embodiment, emitted electromagnetic energy is swept through a bandwidth of about 10 GHz to about 40 GHz for each of a plurality of combinations of the transmit and receive antennas corresponding to respective effective sampling points of the aperture. In other embodiments, the emitted electromagnetic energy is swept through a tighter bandwidth of 10-20 GHz. Other frequency ranges may be used in other embodiments.

Transceiver 50 outputs signals to the transmit antennas to control the emission of electromagnetic energy from the antenna arrays 18 and receives signals from the receive antennas that are indicative of electromagnetic energy received via the antenna arrays 18. Transceiver 50 processes the received signals to provide I, Q outputs to data acquisition system (DAQ) 52 that are indicative of the reflections of the electromagnetic energy received by the receive antennas.

Data acquisition system 20 samples the I, Q signals from the transceiver 50 and outputs digitized samples of the I, Q signals to operator work station 24. The samples outputted from DAQ 52 may be referred to as radar data that are processed by operator workstation 24.

Operator workstation 24 includes an Ethernet switch 54 that implements communications of the workstation 24 with one or more sensor assemblies 20. Workstation 24 additionally includes a computer system 56 that is configured to process outputs from the sensor assemblies 20 including implementing processing operations to focus the radar data received from the sensor assemblies 20. Backprojection is used in some embodiments to focus the radar data and details regarding backprojection focusing at a plurality of different resolutions according to some aspects of the disclosure are described below.

Computer system 56 includes processing circuitry 57 in the illustrated embodiment. Processing circuitry 57 processes the radar data to provide information regarding objects which may be concealed, for example beneath clothing of the individual in the target imaging volume, as discussed in further detail below. In one embodiment, processing circuitry 57 generates images as a result of the processing of the radar data from the scanning of the target imaging volume. The generated images may be displayed, processed, for example by threat detection processing methods, artificial intelligence, and/or stored using storage circuitry (not shown). In some embodiments, the processing circuitry 57 generates a plurality of video images at a given frame rate in real time.

Processing circuitry 57 may comprise circuitry configured to implement programming in at least one embodiment. For example, the processing circuitry 57 may be implemented as one or more processor(s) and/or other structure configured to execute executable instructions including, for example, software and/or firmware instructions. Other example embodiments of processing circuitry 57 include hardware logic, GPU, FPGA, ASIC, state machines, and/or other structures alone or in combination with one or more processor(s). These examples of processing circuitry 57 are for illustration and other configurations are possible.

The focusing of the radar data indicative of electromagnetic energy that is reflected from the target imaging volume creates radar images of the target imaging volume. Some embodiments of computer system 56 include a display 59 that is configured to generate visual representations of the images. In other embodiments, the images are processed by artificial intelligence, processed using automated threat detection, and/or stored for subsequent processing without display of the images.

In one example, computer system 56 is implemented as a Ubuntu 18 computer workstation paired with an Atipa Visione SX426-24G10 dual-socket Xeon GPU server that utilizes six high-end Nvidia GPUs for image processing. The GPU server is housed within a soundproof enclosure for an improved operator experience in some embodiments.

Referring to FIG. 3, one embodiment of a unit cell 30 that may be used in an array 18 is shown. In one implementation, six of the illustrated cells 30 are elevationally stacked in a vertical arrangement above one another to form each of the arrays 18 shown in FIG. 1. Other configurations of the unit cells 30 may be used in other embodiments.

The illustrated unit cell 30 has a plurality of antennas that are separated from one another. In the depicted embodiment, the unit cell 30 is a two-dimensional (2D) antenna array or sub-array having a plurality of transmit antennas 62 and a plurality of receive antennas 64 arranged in a boundary array configuration. Transmit antennas 62 are configured to emit electromagnetic energy towards a target imaging volume and the receive antennas 64 are configured to receive reflections of the electromagnetic energy from the target imaging volume and to output electrical signals corresponding to the electromagnetic energy received by the receive antennas 64.

In the illustrated boundary configuration of unit cell 30, the transmit and receive antennas 62, 64 are provided at a perimeter of the unit cell 30 having a rectangular shape and the perimeter defines a 2D aperture of the unit cell 30. More specifically, the transmit (T) antennas 62 are arranged in vertical subarrays that are positioned at opposing vertical left and right sides of the cell 30 and receive (R) antennas 64 are arranged in horizontal subarrays that are positioned at opposing top and bottom sides of the unit cell 30.

The unit cell 30 is a sparse multistatic array where different combinations of transmit and receive antenna pairs are selected at different moments in time to transmit and receive electromagnetic energy to provide a plurality of respective different virtual effective sampling points 66 at internal locations within the boundary or perimeter of the unit cell 30 corresponding to the 2D aperture of the unit cell 30. The 2D aperture has a first axis a₁ that corresponds to the horizontal extent of the sampling points 66 of the aperture and a second axis a₂ that corresponds to the vertical extent of the sampling points 66 of the aperture.

The use of selected transmit and receive antenna pairs 62, 64 of the described unit cells 30 enables dense and effective sampling of the 2D aperture while requiring relatively few antennas compared to a fully populated two dimensional antenna array. The sampling points 66 are located at approximately midpoints between the selected transmit and receive antenna pairs. The provision of linear arrays of transmit antennas 62 vertically and linear arrays of receive antennas 64 horizontally provides effective sampling points 66 that span a uniform rectangular region of the aperture in the depicted embodiment.

In one embodiment, electromagnetic energy is emitted at plural frequencies of a desired frequency sweep for each of the pairs of transmit and receive antennas 62, 64 and the reflections of electromagnetic energy having the different frequencies are received and processed for each of the sampling points 66 corresponding to the pairs of transmit and receive antennas 62, 64.

In one embodiment, all of the possible different pairs of transmit and receive antennas 62, 64 of the unit cell 30 are selected to provide sampling points 66 across at least substantially the entirety of the 2D aperture of the respective unit cell 30 for scanning of an image or video frame. According to one example method of selecting pairs of transmit and receive antennas, at the initiation of scanning of an image or video frame, a first transmit antenna 62 may be selected to emit the frequency sweep of electromagnetic energy for each of the receive antennas 64 of the unit cell 30. Thereafter, a second transmit antenna 62 is selected to emit the frequency sweep of electromagnetic energy for each of the receive antennas 64 and this process is repeated until all possible pairs of transmit and receive antennas 62, 64 have been selected and responses obtained for each of the sampling points 66 of the unit cell 30.

In one more specific scanning example, the transmit antenna 62 located in the bottom left corner of a unit cell 30 is selected and emits a frequency sweep of electromagnetic energy a plurality of times (i.e., one frequency sweep is emitted from the selected transmit antenna 62 for each receive antenna 64 in the unit cell 30). In this described example, the receive antennas 64 located in the bottom row of the unit cell 30 are sequentially selected from left to right to receive the emitted frequency sweeps from the selected transmit antenna 62. This sequential selection process is repeated for the receive antennas 64 located in the top row of the unit cell 30 to receive the emitted frequency sweep from the selected transmit antenna 62. Following all of the receive antennas 64 receiving an emitted frequency sweep from the transmit antenna 62 located in the bottom corner, the next transmit antenna 62 above the bottom left corner is selected to emit a plurality of frequency sweeps to be received by the receive antennas 64 in the top and bottom of the unit cell 30 that are selected one-by-one as mentioned above. Following transmission of the frequency sweeps from the transmit antennas 62 located on the left of the unit cell 30, the above-described process is repeated for the transmit antennas 62 located on the right of the unit cell 30. This example scanning method generates the effective sampling points 66 for the unit cell 30 that are substantially uniformly spaced at substantially the same distance from one another and at locations across substantially an entirety of the 2D aperture of the unit cell 30 as shown in FIG. 3. A vertical gap 61 and a horizontal gap 63 within the 2D aperture of the unit cell 30 are shown due to a design assumption that the antennas require a minimum physical separation distance from one another (e.g., antennas are separated by a distance of 20 mm).

The example antenna boundary array configuration of FIG. 3 allows for extremely dense sampling and thus provides superb image quality without use of a mechanically scanned aperture nor any motion tracking technology.

The apertures of multiple unit cells 30 may be combined to define larger 2D apertures of the antenna system 12, for example combining the 2D apertures of six vertically arranged unit cells 30 to form a larger 2D aperture of an antenna array 18 or antenna system 12 of the embodiment shown in FIG. 1. Additional details regarding example hardware configurations of imaging systems and switched antenna modules that may be utilized to form unit cells 30, antenna arrays 18, and multicolumn antenna architectures are discussed in a first US patent application filed on the same date as this US patent application, having Attorney Docket No. 31915-E A (BA4-0868), titled “Imaging Systems and Imaging Methods,” and including inventors David M. Sheen, A. Mark Jones, Jonathan R. Tedeschi, Richard Trevor Clark, Maurio B. Grando and Ryan C. Conrad, and a second US patent application filed on the same date as this US patent application, having Attorney Docket No. 31915-E B (BA4-0966), titled “Imaging Systems and Imaging Methods,” and including inventors David M. Sheen and Richard Trevor Clark, and the teachings of both of these US patent applications are incorporated herein by reference.

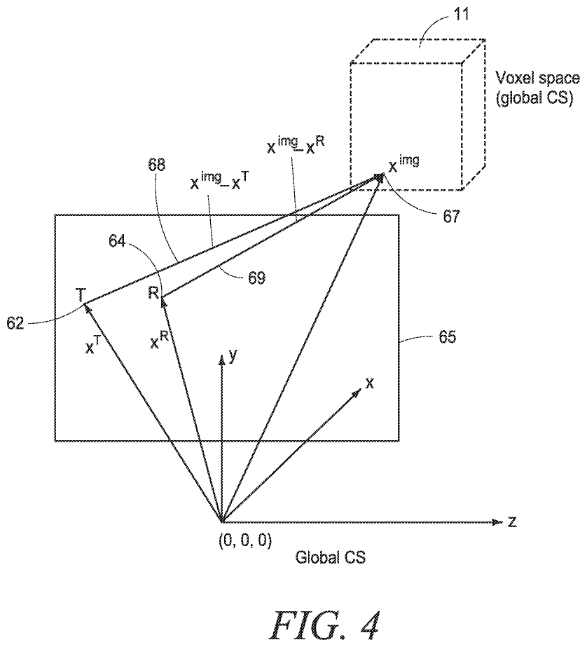

Referring to FIG. 4, an illustrative representation is shown of scanning operations where transmit and receive antennas of antenna system are electronically scanned over a two-dimensional 2D multistatic aperture 65 that may be formed by one or more unit cells 30. A global coordinate system (global CS) is used for backprojection focusing and the coordinate system of the voxel space or target imaging volume 11 is used as the global coordinate system in one embodiment.

Although a transmit antenna 62 and a receive antenna 64 of one pair are shown in FIG. 4, it is understood that additional different pairs of transmit and receive antennas of the antenna system are selected to transmit electromagnetic energy towards target imaging volume 11 and receive electromagnetic energy reflected from volume 11 at different times. The selection of different pairs of transmit and receive antennas 62, 64 at different times for scanning results in a plurality of sampling points 66 over at least substantially an entirety of the multistatic aperture 65.

In one embodiment, a target may be either stationary within or moving through target imaging volume 11 as different pairs of transmit and receive antennas are scanned. One of a plurality of voxels 67 (i.e., x^{img}) is shown receiving electromagnetic energy 68 from transmit antenna 62 of a selected pair of antennas and reflecting electromagnetic energy 69 to receive antenna 64 of the selected pair of antennas.

The selection of different antenna arrays during scanning provides the scanned aperture 65 which includes different transmit and receive combinations of the antennas of the antenna arrays 18 whose effective phase centers (i.e., effective sampling points) span the extent of the aperture 65 as mentioned above.

The discussion proceeds below with respect to example operations performed by the processing circuitry of workstation 24 with respect to the radar data to perform range-domain multistatic backprojection focusing to generate images of a target imaging volume. In one embodiment, the processing circuitry of the imaging system accesses and processes the radar data using backprojection 3D image reconstruction. As discussed in US Patent Publication No. 2020/0319331 A1, the teachings of which are incorporated herein by reference, one parameter to perform back-projection focusing of the reconstruction is the effective range from a selected pair of transmit and receive antennas to a given image voxel of the target imaging volume, which is defined here as one-half the round-trip distance. For a single voxel, a frequency-domain back-projection algorithm can be expressed as:

$$v ( {x}^{i m g} ) = {\sum }_{{aperture}_{\{ {a}_{1} , {a}_{2} \}}} w ( {a}_{1} , {a}_{2} ) {\sum }_{f} S ( {a}_{1} , {a}_{2} , f ) {e}^{j 2 k r} \qquad Eq . 1$$

where v(x^{img}) is the complex image amplitude or intensity at an image position or voxel x^{img} of the target imaging volume 11, S (a₁, a₂, f) is the complex radar phase-history data collected over aperture dimensions a₁, a₂ and f is frequency. An aperture weighting term w(a₁, a₂) is used here to provide amplitude weighting of calculated intensities of the voxels to reduce side lobes or other artifacts in the image and which is discussed in further detail below. Note that S and w are both typically discrete multidimensional arrays rather than continuous functions in one embodiment. The conjugate phase term in this expression is e^{j2kr} where k=2πf/c, c is the speed of light, and

$$r = ( \vert {x}^{i m g} - {x}^{T} \vert + \vert {x}^{i m g} - {x}^{R} \vert ) / 2 \qquad Eq . 2$$

In this expression, x^{T} is the location of the transmitting antenna upon the antenna array and x^{R} is the location of the receiving antenna upon the antenna array, x^{img} is the image voxel location of the target imaging volume, and the round-trip distance is divided by 2 so that the range (r) is an equivalent or “effective” one-way distance to the voxel of the target from a selected pair of the transmit and receive antennas. This is done for consistency with monostatic or quasi-monostatic radar systems.

For 3D imaging, the above processing has a computational burden of O(N⁶) upon the processing circuitry where N is the nominal dimension of each axis of the voxel space, frequency, and aperture dimensions.

As is further discussed in US Patent Publication No. 2020/0319331 A1, the order of the process can be reduced to O(N⁵) by transforming the radar data from the frequency domain to the range domain and as also discussed described below. In the described embodiment, the radar data has a complex image amplitude or intensity v(x^{img}) at an image position or voxel x^{img} of the range domain back-projection expressed in Eq. 3.

As also discussed in US Patent Publication No. 2020/0319331 A1, there is a fast phase variation of e^{j2k}^{_{c}}^{r} where k_{c}= (k₁+k₂)/2 is the center wavenumber and k₂=2π fₛₜₒₚ is the final wavenumber since the range response is queried during summation using interpolation and this phase variation may lead to errors or require that the range response be overly finely sampled. This variation can be largely removed by demodulating the data with a e^{-j2k}^{_{c}}^{r} term and subsequently remodulating the data with a e^{+j2k}^{_{c}}^{r} term. The complex image amplitude or intensity v(x^{img}) at an image position or voxel x^{img} of the range domain back-projection can be expressed as

$$v ( {x}^{i m g} ) = {\sum }_{{a}_{1}} {\sum }_{{a}_{2}} w ( {a}_{1} , {a}_{2} ) s ( {a}_{1} , {a}_{2} , r ) {e}^{j 2 {k}_{{c}^{r}}} \\ where \qquad Eq . 3$$

$$s ( {a}_{1} , {a}_{2} , r ) = \{ i F F {T}_{f} ( S ( {a}_{1} , {a}_{2} , f ) ) {e}^{j 2 {k}_{1} {r}_{n}} {e}^{- j 2 {k}_{c} {r}_{n}} \} \qquad Eq . 4$$

is the demodulated range response computed by performing an iFFT of the phase history on the frequency axis, applying a phase term of e^{j2k}^{₁}^{r} e^{-j2k}^{_{c}}^{r}, and using interpolation (usually linear) to compute the value at range r, which is

$$r = ( \vert {x}^{i m g} - {x}^{T} \vert + \vert {x}^{i m g} - {x}^{R} \vert ) / 2 \qquad Eq . 5$$

The complete image is formed by evaluating Eq. 3 over the full 3D set of voxel locations and the overall order of the computation performed by the processing circuitry 57 is reduced from O(N⁶) to O(N⁵).

In one embodiment, locations and orientations of the transmitters and receivers of one or more unit cells of the antenna system are defined in an array path sequence file shown in Table A. This array has NTR rows, which is the number of different combinations of transmit and receive antennas, or elements, in the array. The disclosed array path sequence file is used to keep track of the transmit and receive antenna positions (e.g., x, y, z) and orientations (e.g., unit vectors with x, y, z components) for each virtual sampling point of the aperture. The inclusion of the information regarding the transmit and receive antennas in an array path sequence file is convenient as information regarding the sampling points is used many times during the processing and focusing of the radar data.

TABLE A
Array Path Sequence (APS) File

| Column | Description |
|---|---|
| 0 | TR index number |
| 1 | T index |
| 2:5 | T position (x, y, z) |
| 5 | R index |
| 6:9 | R position (x, y, z) |
| 9:12 | Phase Center (midpoint of T and R) (x, y, z) |
| 12:15 | T orientation vector (nₓ, n_{y}, n_{z}) |
| 15:19 | R orientation vector (nₓ, n_{y}, n_{z}) |

In the case of scanning a person or other object, the radar image is sparse, or in other words, generated images of the 3D volume only have meaningful data above a certain threshold (e.g., 30 dB below the maximum) in a relatively small percentage of the voxels. If the specific voxels containing sufficient intensity data, or in other words, the precise locations of scatterers in the target imaging volume were known a-priori, then the focusing (i.e., summation) may be performed for only those voxels with the sufficient intensity data which results in a speed up of the focusing of the radar data by an order of magnitude or more.

The discussion proceeds with respect to an example method of sparse multi-resolution image reconstruction that may be performed upon the radar data by the processing circuitry of the computer system in accordance with some aspects of the disclosure.

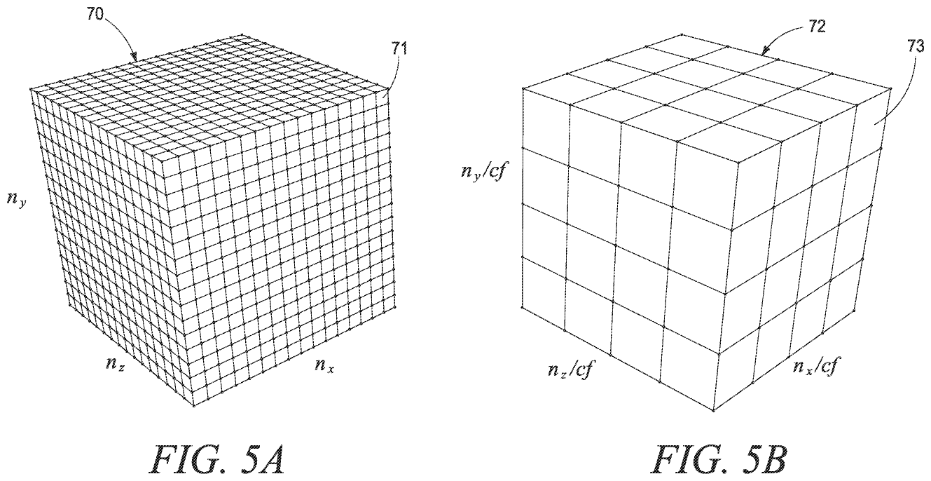

Initially, a three-dimensional (3D) fine voxel grid is defined that corresponds to the target imaging volume of the imaging system. Referring to FIG. 5A, an example fine voxel grid 70 is shown with dimensions (nₓ, n_{y}, n_{z}) and includes a plurality of fine voxels 71.

Then, a lower resolution or coarse voxel grid 72 of the fine voxel grid 70 is defined to determine coarse voxels of interest (and which utilizes less computation compared with focusing the radar data for the fine voxel grid 70). Referring to FIG. 5B, a 3D lower resolution coarse voxel grid 72 is defined by setting a coarseness factor (cf) to down sample the fine voxel grid 70. The coarse voxel grid 72 occupies the same volume as the fine voxel grid 70 and has dimensions (nₓ/cf, n_{y}/cf, n_{z}/cf) and includes a plurality of coarse voxels 73. Fine voxels 71 have increased resolution and decreased volume compared with the resolution and volume of coarse voxels 73.

Equations 3-5 of the above-described range-domain multistatic backprojection focusing may be used to initially focus the radar data providing initial focused data in the form of intensity values for coarse voxels 73 of the coarse voxel grid 72 of the target imaging volume. In one embodiment, the radar data is focused over the entire coarse voxel grid 72 corresponding to at least substantially an entirety of the target imaging volume. The focusing creates a low resolution image including an intensity value for each coarse voxel 73 of coarse voxel grid 72 in the described embodiment. The computation time for focusing of all of the coarse voxels 73 of the coarse voxel grid 72 is (cf)\hat{ }3 lower than the focusing of all of the fine voxels 71 of fine voxel grid 70.

In some embodiments, different coarseness factors cfₓ, cf_{y}, cf_{z} are used for different x, y, z dimensions and the computation is accordingly reduced by (cfₓ cf_{y} cf_{z}).

The initial focused data is further processed to select coarse voxels that correspond to scatterers in the target imaging volume. In one embodiment, the processing circuitry selects or identifies coarse voxels having intensity values greater than a threshold intensity, such as 30 dB below a maximum intensity as mentioned above. For example, the intensity values of the coarse voxels 73 are compared with the threshold and the coarse voxels 73 having intensity values greater than the threshold are selected.

In one embodiment, the selected coarse voxels 73 having sufficient intensity data are used to identify or determine the fine voxels 71 of the higher resolution fine voxel grid 70 that correspond to the selected coarse voxels 73 and will be focused in subsequent processing. The selected coarse voxels 73 correspond to or define a sub-volume of the target imaging volume and coarse voxel grid 72.

Thereafter, the selected coarse voxels are used to identify a plurality of fine voxels 71 of the fine voxel grid 70 of the target imaging volume that correspond to the selected coarse voxels. In one embodiment, the selected coarse voxels define a sub-volume of the target imaging volume and the identified fine voxels correspond to and are enclosed in the same sub-volume of the target imaging volume. Accordingly, the identified fine voxels 71 occupy a sub-volume of fine voxel grid 70 that corresponds to the sub-volume of the identified or selected coarse voxels 73 of the coarse voxel grid 72 in the described embodiment. The selected coarse voxels 73 of the sub-volume of the coarse voxel grid 72 may be considered to be translated to the identified fine voxels 71 of the sub-volume of the fine voxel grid 70 in one embodiment.

Thereafter, the range-domain multistatic backprojection focusing of equations 3-5 is performed for the identified or selected fine voxels 71 to generate a final high-resolution image of the target imaging volume. The above-described focusing operates to focus the radar data to a plurality of discrete points in space (i.e., the centers of the voxels) and the resolution may be adjusted by adjusting the distance between focused positions during the initial coarse processing of the radar data for the coarse voxels and subsequent processing of the radar data for the identified or selected fine voxels of the dilated sub-volume.

The subsequent focusing of the radar data is performed for an increased number of closely-spaced fine voxels for the identified sub-volume compared with the number of coarse voxels of the sub-volume that were focused during the initial coarse focusing of the radar data. In one embodiment, the coarse voxels 71 each have a size of approximately 15 mm and the fine voxels 73 have a size of approximately 5 mm with a coarseness factor cf=3.

According to some embodiments, a dilation operation may be performed upon the selected coarse voxels 73 of the low resolution image prior to the translation to identify the fine voxels 71 in an attempt to avoid missing scatterers within the target imaging volume because of the coarseness of the coarse voxel grid 72 of the target imaging volume. One example dilation method applies a kernel of size (kₓ, k_{y}, k_{z}) to each of the selected coarse voxels and that identifies additional coarse voxels 73 that were not previously identified nor selected following the initial processing of the radar data for the coarse voxels 73 (i.e., due to having insufficient intensity values below the threshold). In one specific embodiment, binary dilation is implemented using a (3,3,3) dilation kernel to identify additional coarse voxels 73 in three dimensions that are immediately adjacent to each of the selected coarse voxels 73. Larger kernels may be used in other embodiments to select additional coarse voxels 73 in three dimensions that are immediately adjacent to as well as adjacent to and within a specified distance from a selected coarse voxel 73.

In one embodiment, the initially selected coarse voxels and the additional coarse voxels identified by the above-described dilatation are used to define the sub-volume of the coarse voxel grid 72 that is translated to the sub-volume of the fine voxel grid 70 to identify the fine voxels of the fine voxel grid 70 that are enclosed in the sub-volume of the fine voxel grid 70 and are to be focused using the above-described backprojection.

The described dilation provides ample padding around initially detected scatterers in the target imaging volume to generate a complete high resolution image while taking advantage of the efficiencies of multi-resolution processing of sparse radar images. In particular, the range-domain multistatic backprojection focusing is performed upon the radar data for the identified fine voxels of the dilated sub-volume providing an image of the target imaging volume at the desired increased resolution that may be displayed and/or processed to identify and evaluate possible threats included in the image. Accordingly, the processing circuitry focuses the radar data for only the identified fine voxels of the dilated sub-volume and which correspond to less than an entirety of the fine voxel grid 70 and target imaging volume and the processing circuitry does not focus radar data for fine voxels that were not enclosed in the dilated sub-volume of the fine voxel grid 70. In one embodiment, the processing circuitry focuses the same set of radar data using equations 3-5 during the multi-resolution focusing to obtain intensity values for the coarse voxels 73 and fine voxels 71.

The fine voxel grid may be referred to as a final voxel grid as it has a resolution corresponding to a final generated high resolution image that is displayed or further processed to detect concealed threats upon a person or other target. In other embodiments, backprojection may be performed perhaps iteratively on the radar data for one or more additional increased resolutions (i.e., in addition to focusing at two different resolutions as described above) if desired in an effort generate final images of further increased resolution.

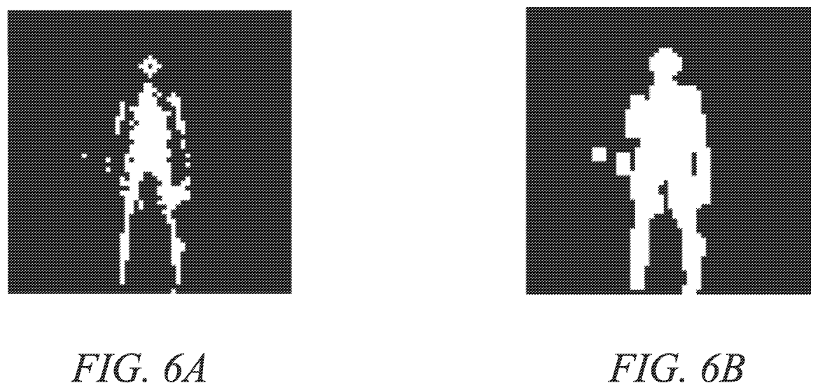

Referring to FIG. 6A, a 2D representation is shown of all of the coarse voxels of the coarse voxel grid that have a value above a given threshold. The 2D representation of FIG. 6A results from focusing radar data of an entirety of the target imaging volume at a lower coarse resolution.

FIG. 6B shows a 2D representation of all of the coarse voxels of the coarse voxel grid that have been selected after binary dilation processing. The 2D representation of FIG. 6B results from the application of a (3,3,3) dilation kernel to the selected coarse voxels having sufficient intensity values greater than the threshold. The illustrated 2D representations correspond to sub-volumes defined by the initially selected coarse voxels in FIG. 6A and both the initially selected coarse voxels and the additional coarse voxels selected by the application of the dilation kernel in FIG. 6B.

Example methods of processing radar data to generate images of a target imaging volume discussed herein have some advantages over other focusing techniques. In some embodiments, the focused images do not have artifacts since the voxels containing sufficient intensity data are focused using the full range-domain backprojection algorithm without additional interpolation steps. In addition, there is minimal penalty for using a relatively large target imaging volume due to the exploitation of the sparse nature of the radar images. The dilation operations discussed above according to some embodiments and limited resolution of radar image systems also reduce the chance that scatterers or objects are missed.

In addition, the sparse nature of resultant images may be exploited to identify locations of scatterers within the target imaging volume without additional hardware by performing multi-resolution reconstruction upon obtained radar data as described according to example embodiments of the disclosure. The sparse multi-resolution backprojection techniques discussed herein may be utilized in microwave and millimeter-wave imaging systems in a straightforward manner. Performing the disclosed sparse reconstruction over the volume of interest enables the time for reconstruction to be drastically reduced thereby enabling near-real-time operation while maintaining optimal image fidelity in some implementations.

The example image reconstruction methods disclosed herein permit effective operation in the near field of the arrays by calculating the transmit to receive path lengths between the transmit and receive antennas using the exact locations of the transmit and receive antennas as opposed to replacement of the transmit and receive antenna locations with an approximation in the form of an equivalent sample position, or phase center. Some of the imaging systems and methods disclosed herein make no such approximations and therefore enable close-range operation in the near field of the antenna system (e.g., in ranges comparable to the maximum separation distance from a transmit antenna to a receive antenna, or a depth of 50-100 cm from the antenna system in some embodiments).

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended aspects appropriately interpreted in accordance with the doctrine of equivalents.

Further, aspects herein have been presented for guidance in construction and/or operation of illustrative embodiments of the disclosure. Applicant(s) hereof consider these described illustrative embodiments to also include, disclose and describe further inventive aspects in addition to those explicitly disclosed. For example, the additional inventive aspects may include less, more and/or alternative features than those described in the illustrative embodiments. In more specific examples, Applicants consider the disclosure to include, disclose and describe methods which include less, more and/or alternative steps than those methods explicitly disclosed as well as apparatus which includes less, more and/or alternative structure than the explicitly disclosed structure.

What is claimed is:

1. An imaging system comprising:
an antenna system comprising a plurality of transmit antennas and a plurality of receive antennas, and wherein the transmit antennas are configured to emit electromagnetic energy towards a target imaging volume and the receive antennas are configured to receive reflections of the electromagnetic energy from the target imaging volume;
a transceiver coupled with the antenna system, and wherein the transceiver is configured to control the emission of the electromagnetic energy from the transmit antennas and to output signals that are indicative of the reflections of the electromagnetic energy received via the receive antennas;
a data acquisition system configured to sample the signals from the transceiver and output radar data corresponding to the signals; and
processing circuitry configured to:
access the radar data from the data acquisition system;
first focus the radar data providing first focused data for a plurality of a first voxels of the target imaging volume, and wherein the first voxels have a first resolution;
use the first focused data to select some of the first voxels;
use the selected first voxels to identify a plurality of second voxels of the target imaging volume, and wherein the second voxels have a second resolution that is increased compared with the first resolution; and
second focus the radar data providing second focused data for the identified second voxels, wherein the second focused data comprises an image of the target imaging volume.

2. The system of claim 1 wherein the processing circuitry is configured to first focus the radar data for the first voxels which correspond to at least substantially an entirety of target imaging volume, and the processing circuitry is configured to second focus the radar data for the identified second voxels which correspond to less than the entirety of target imaging volume.

3. The system of claim 1 wherein the first focused data comprises a plurality of intensity values for the first voxels, and the processing circuitry is configured to select the some of the first voxels having intensity values greater than a threshold.

4. The system of claim 1 wherein the some of the first voxels correspond to a sub-volume within the target imaging volume, and the second voxels correspond to at least substantially an entirety of the sub-volume.

5. The system of claim 1 further comprising a display configured to use the second focused data to generate a visual representation of the image.

6. The system of claim 1 wherein the processing circuitry is configured to use the some of the first voxels to identify a plurality of additional first voxels that were not selected, use the additional first voxels to identify a plurality of additional second voxels, and wherein the processing circuitry is configured to perform the second focusing comprising second focusing the radar data providing the second focused data for the additional second voxels.

7. The system of claim 6 wherein the processing circuitry is configured to identify the additional first voxels that are adjacent to the some of the first voxels.

8. The system of claim 6 wherein the processing circuitry is configured to use the some of the first voxels to identify the additional first voxels in three dimensions.

9. The system of claim 1 wherein the processing circuitry is configured to not focus the radar data for others of the second voxels that were not identified.

10. The system of claim 1 wherein the processing circuitry focuses the same set of radar data during execution of the first and second focusing.

11. An imaging system comprising:
processing circuitry configured to access radar data resulting from reflection of electromagnetic energy from a target imaging volume;
first focus the radar data to provide first focused data for at least substantially an entirety of the target imaging volume;
use the first focused data to identify a sub-volume of the target imaging volume; and
second focus the radar data to provide second focused data for the sub-volume of the target imaging volume, and wherein the second focused data has increased resolution compared with the first focused data and the second focused data comprises an image of the target imaging volume.

12. The system of claim 11 wherein the first focused data comprises a plurality of intensity values for a plurality of voxels, and the processing circuitry is configured to identify the sub-volume comprising some of the voxels having intensity values greater than a threshold.

13. The system of claim 11 wherein the processing circuitry is configured to focus the same set of radar data during execution of the first and second focusing.

14. An imaging method comprising:
emitting electromagnetic energy towards a target imaging volume;
receiving the electromagnetic energy reflected from the target imaging volume;
generating radar data indicative of the received electromagnetic energy;
first focusing the radar data to provide first focused data at a first resolution of the target imaging volume;
using the first focused data to identify a sub-volume within the target imaging volume; and
second focusing the radar data corresponding to the sub-volume to provide second focused data at a second resolution of the target imaging volume, wherein the second resolution is increased compared with the first resolution and the second focused data comprises an image of the target imaging volume.

15. The method of claim 14 wherein the first focused data comprises a plurality of intensity values for a plurality of first voxels at the first resolution, wherein the using the first focused data comprises selecting some of the first voxels having intensity values greater than a threshold, and wherein the sub-volume corresponds to the some of the first voxels.

16. The method of claim 14 wherein the first focused data comprises data for a plurality of first voxels having the first resolution and the second focused data comprises data for a plurality of second voxels having the second resolution.

17. The method of claim 16 wherein the using the first focused data comprises using the first focused data to select some of the first voxels, and wherein the sub-volume corresponds to a volume defined by the some of the first voxels.

18. The method of claim 17 further comprising using the some of the first voxels to identify a plurality of additional first voxels that were not selected, and wherein the sub-volume additionally corresponds to a volume defined by the additional first voxels.

19. The method of claim 14 further comprising displaying the second focused data to generate a visual representation of the image.

20. The method of claim 14 wherein the second focusing comprises second focusing only the radar data that corresponds to the sub-volume.

21. The method of claim 14 wherein the first and second focusings individually comprise focusing the same set of data.
